(19) United States
(12) Patent Application Publication
(10) Pub. No.: US 2024/0285187 A1
(43) Pub. Date: Aug. 29, 2024

(54) SNIFF DETECTION AND ARTIFACT DISTINCTION FROM ELECTROMYOGRAPHY AND ACCELEROMETER SIGNALS

(71) Applicant: KONINKLIJKE PHILIPS N.V., EINDHOVEN (NL)

(72) Inventors: JAKOB VAN DE LAAR, OOSTERHOUT (NL); JENS MUEHLSTEFF, AACHEN (DE); MARIAN DEKKER, NOORD BRABANT (NL)

(21) Appl. No.: 18/573,480

(22) PCT Filed: Jun. 27, 2022
(86) PCT No.: PCT/EP2022/067521

§ 371 Date: Dec. 22, 2023

Related U.S. Application Data
(60) Provisional application No. 63/216,269, filed on Jun. 29, 2021.

(51) Int. Cl. A61B 5/085; A61B 5/00; A61B 5/11; A61B 5/113; A61B 5/389

(52) U.S. Cl. CPC A61B 5/085; A61B 5/1126; A61B 5/113; A61B 5/389; A61B 5/7221; A61B 5/7225; A61B 2562/0219; A61B 2562/04; A61B 2562/06

(57) ABSTRACT

Non-invasive systems and methods for quantifying respiratory muscle effort (RME) (or breathing effort, work-of-breathing) are provided. The systems and methods utilize simultaneously measured EMG and accelerometer signals. The measured EMG signal is preprocessed to produce both a signal accentuating regular breathing activity and a signal accentuating sniff activity (deep, sharp inhalations). Time intervals of candidate sniffs are determined from the preprocessed EMG signals. The measured accelerometer signal is preprocessed to produce multiple signals accentuating either upper or lower frequency band activity. Features of the preprocessed EMG and accelerometer signals corresponding to the time intervals of candidate sniffs are analyzed to determine if the candidate sniffs constitute actual sniffs or artifacts. Subsequently, after maximum sniff effort has been identified, RME is quantified by the ratio of the mean of the maxima of regular breathing activity to the value of the maximum sniff effort.

### BACKGROUND OF THE INVENTION

### 1. Field of the Invention

The disclosed concept pertains to methods and systems for quantifying the breathing effort (i.e. respiratory muscle effort, or “work-of-breathing”) of a patient and, in particular, to methods and systems for distinguishing electromyography (EMG) signals indicative of sniffing (i.e. deep, sharp inhalation) efforts from EMG signal artifacts.

### 2. Description of the Related Art

Electromyography (EMG) can be used to assess the respiratory status of a patient by deducing the activity of muscles involved in respiration, such as the intercostal spaces on bilateral sides of the sternum (parasternal) or the abdominal area close to the diaphragm. While respiration rate can easily be measured non-invasively, respiration rate alone is not indicative of the effort output by a patient while breathing. By comparison, EMG measurements of the inspiratory muscles are indicators of the balance between respiratory muscle load and respiratory muscle capacity. EMG signals provide a non-invasive method for obtaining an objective measure of breathing effort. In particular, respiratory EMG activity as measured during inhalations represents the neural respiratory drive, which is a signal that the brain outputs to the respiratory muscles and an indicator of the balance between respiratory muscle load and respiratory muscle capacity.

Objective measures of respiratory muscle activity derived from EMG signals are considered to be important for monitoring the respiratory status of patients, such as inpatients with chronic obstructive pulmonary disease (COPD). In addition to EMG measurements of regular breathing activity occurring naturally when the patient is relaxed, EMG measurements of sniffs are highly informative in assessing the respiratory status of patients as well. Sniffs are deep and sharp inhalations (maximum effort maneuvers). However, isolating sniff activity in EMG signals is not straightforward, as signal artifacts due to non-respiratory activity give rise to strong activity in the EMG signal that often resembles sniff activity. Such signal artifacts can for example be caused by patient movements or disturbances of electrodes due to pressure on the connected wires.

The ability to reliably detect sniffs and distinguish sniffs from artifacts directly impacts the estimates of respiratory muscle activity from EMG signals and the respiratory status of a patient. Independent measurements of breathing activity with devices such as nasal cannulas or esophageal electrodes provide additional breathing data that can be compared to the EMG signal in order to distinguish sniffs from signal artifacts on an EMG waveform. However, nasal cannulas, esophageal electrodes, and other similar devices are highly invasive and can be cumbersome or even a burden to patients.

Accordingly, there is room for improvement in methods and systems used to differentiate sniffs from other activity in EMG signals.

### SUMMARY OF THE INVENTION

Accordingly, it is an object of the present invention to provide, in one embodiment, a method for quantifying respiratory muscle effort (i.e. breathing activity, work-of-breathing) of a patient during a breathing interval that includes: measuring respiratory muscle activity of the patient with a number of EMG electrodes, measuring acceleration in a plurality of planes of the patient's thorax concurrently with respiratory activity with an accelerometer, receiving a raw EMG signal measured by the EMG electrodes and raw accelerometer signals measured by the accelerometer with a controller, producing a number of preprocessed EMG signals by preprocessing the raw EMG signal with the controller, producing a number of preprocessed accelerometer signals by preprocessing the raw accelerometer signals with the controller, identifying portions of the number of preprocessed EMG signals as candidate sniffs with the controller, determining a plurality of EMG-derived features with the controller from the number of preprocessed EMG signals associated with time intervals of the candidate sniffs, determining a plurality of accelerometer signal features with the controller from the number of preprocessed accelerometer signals associated with time intervals of the candidate sniffs, comparing the plurality of EMG-derived and accelerometer signal features to a plurality of sniff detection threshold values with the controller, classifying the candidate sniffs as confirmed sniffs with the controller if results of the comparing indicate sniff activity, and quantifying a respiratory muscle effort of the patient with the controller by comparing a number of attributes of the number of preprocessed EMG signals to a number of attributes of the confirmed sniffs.

The method may further comprise using the controller to: identify local regular breathing EMG maxima associated with regular breathing in the number of preprocessed EMG signals, determine the mean of the local regular breathing EMG maxima, and identify a maximum sniff value in the number of preprocessed EMG signals associated with the confirmed sniffs. Quantifying the respiratory muscle effort may comprise comparing the mean of the local regular breathing EMG maxima to the maximum sniff value. The method may further comprise using the controller to: produce a regular breathing EMG signal by preprocessing the raw EMG signal to accentuate regular breathing activity and minimize artifacts in the raw EMG signal; produce a sniff EMG signal by preprocessing the raw EMG signal to accentuate sniff activity in the raw EMG signal, identify the local regular breathing EMG maxima in the regular breathing EMG signal, identify local regular breathing EMG minima in the regular breathing EMG signal, and identify the maximum sniff value in the sniff EMG signal. Quantifying the respiratory muscle effort of the patient may comprise finding a ratio of the mean of the local EMG maxima to the maximum sniff value.

The method may further comprise using the controller to: identify, for each candidate sniff, a bump of the candidate sniff such that all values of the sniff EMG signal in the bump are greater than or equal to a predetermined threshold sniff value; identify a midpoint in each bump, said midpoint being a median such that an area under a curve of a left half of the bump is equal to an area under a curve of a right half of the bump; calculate an offset value by linearly interpolating between the local sniff EMG minimum immediately preceding the bump and the local sniff EMG minimum immediately following the bump; determine an amplitude of the bump by finding the difference between a maximum sniff EMG value in the bump and the offset value; and classify the candidate sniff as an artifact if a number of predetermined amplitude conditions are indicative of artifact activity. The method may further comprise using the controller to: determine a first asymmetry feature of the bump by finding the ratio of the mean of the local sniff EMG minimum immediately preceding the bump and the local sniff EMG minimum immediately following the bump to the amplitude; determine a second asymmetry feature of the bump by finding a first difference between the local sniff EMG minimum immediately preceding the bump and the local sniff EMG minimum immediately following the bump, by finding a second difference between the maximum sniff value in the bump and the local sniff EMG minimum immediately preceding the bump, by finding a third difference between the maximum sniff value in the bump and the local sniff EMG minimum immediately following the bump, and by finding the ratio of the first difference to the lesser of the second difference and the third difference; determine a third asymmetry feature of the bump by determining a skewness of the bump; and classify the candidate sniff as an artifact if a number of predetermined asymmetry conditions are indicative of artifact activity.

The method may further comprise using the controller to: low-pass filter, rectify, and smooth the raw accelerometer signals to produce a lower frequency band power signal; high-pass filter, rectify, and smooth the raw accelerometer signals to produce an upper frequency band power signal; sum the lower frequency band power signal and upper frequency band power signal to produce a summed frequency band power signal; determine a first ratio of the upper frequency band power signal to the summed frequency band power signal for a first axis of the accelerometer during the time interval associated with each of the candidate sniffs; determine a second ratio of the upper frequency band power signal to the summed frequency band power signal for a second axis of the accelerometer during the time interval associated with each of the candidate sniffs; compare the first ratio and the second ratio to a predetermined frequency band ratio; and qualify as an artifact any of the candidate sniffs for which the first ratio and the second ratio exceeds the predetermined frequency band ratio.

The method may further comprise using the controller to: high-pass filter, rectify, and smooth the raw accelerometer signals to produce an upper frequency band power signal; determine the number of times the upper frequency band power signal crosses a predetermined threshold value during the time interval associated with each of the candidate sniffs; and qualify as an artifact any of the candidate sniffs for which the number of times the upper frequency band power signal crosses the predetermined threshold exceeds a predetermined number of crossings during the time interval associated with the candidate sniff. The method may further comprise using the controller to: low-pass filter the raw accelerometer signals to produce a lower frequency band signal, high-pass filter the raw accelerometer signals to produce an upper frequency band signal, determine the standard deviation of the lower frequency band signal and the higher frequency band signal, and qualify as an artifact any of the candidate sniffs for which the standard deviation exceeds a predetermined value.

In another embodiment, a system for quantifying respiratory effort of a patient during a breathing interval includes: a number of EMG electrodes configured to measure respiratory muscle activity of the patient, an accelerometer configured to measure acceleration of a plurality of axes of the patient's thorax, and a controller. The controller is configured to: receive a raw EMG signal measured by the EMG electrodes and raw accelerometer signals measured by the accelerometer, produce a number of preprocessed EMG signals by preprocessing the raw EMG signal, produce a number of preprocessed accelerometer signals by preprocessing the raw accelerometer signals, identify portions of the number of preprocessed EMG signals as candidate sniffs, determine a plurality of EMG-derived features from the number of preprocessed EMG signals associated with time intervals of the candidate sniffs, determine a plurality of accelerometer signal features from the number of preprocessed accelerometer signals associated with time intervals of the candidate sniffs, compare the plurality of EMG-derived and accelerometer signal features to a plurality of sniff detection threshold values, classify the candidate sniffs as confirmed sniffs or as signal artifacts based on comparisons of the plurality of EMG-derived features and accelerometer signal features to the plurality of sniff detection threshold values, and quantify a respiratory muscle effort of the patient by comparing a number of attributes of the number of preprocessed EMG signals to a number of attributes of the confirmed sniffs.

The controller of the system may be further configured to: identify local regular breathing EMG maxima associated with regular breathing in the number of preprocessed EMG signals, determine the mean of the local regular breathing EMG maxima, identify a maximum sniff value in the number of preprocessed EMG signals associated with the confirmed sniffs, and quantify the respiratory muscle effort by comparing the mean of the local EMG maxima to the maximum sniff value. The controller of the system may be further configured to: produce a regular breathing EMG signal by preprocessing the raw EMG signal to accentuate regular breathing activity and minimize artifacts in the EMG signal, produce a sniff EMG signal by preprocessing the raw EMG signal to accentuate sniff activity in the EMG signal, identify local EMG maxima in the regular breathing EMG signal, and identify the maximum sniff value in the sniff EMG signal. Comparing the mean of the local EMG maxima to the maximum sniff value may comprise finding the ratio of the mean of the local EMG maxima to the maximum sniff value.

The controller of the system may be further configured to: identify, for each candidate sniff, a bump of the candidate sniff such that all values of the sniff EMG signal in the bump are greater than or equal to a predetermined threshold sniff value; identify a midpoint in each bump, said midpoint being a median such that an area under a curve of a left half of the bump is equal to an area under a curve of a right half of the bump; determine, at the midpoint of each bump, an offset value by linearly interpolating between the local sniff EMG minimum immediately preceding the bump and the local sniff EMG minimum immediately following the bump; determine an amplitude of the bump by finding the difference between a maximum sniff EMG value in the bump and the offset value; and classify the candidate sniff as an artifact if a number of predetermined amplitude conditions are indicative of artifact activity.

The controller of the system may be further configured to: high-pass filter, rectify, and smooth the raw accelerometer signals to produce an upper frequency band power signal; determine the number of times the upper frequency band power signal crosses a predetermined threshold value during the time interval associated with each of the candidate sniffs; and qualify as an artifact any of the candidate sniffs for which the number of times the upper frequency band power signal crosses the predetermined threshold exceeds a predetermined number of crossings during the time interval associated with the candidate sniff. The system may be further configured to: low-pass filter the raw accelerometer signals to produce a lower frequency band signal, high-pass filter the raw accelerometer signals to produce an upper frequency band signal, the standard deviation of the lower frequency band signal and the higher frequency band signal, and qualify as an artifact any of the candidate sniffs for which the standard deviation exceeds a predetermined value.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1A is a perspective view of the anterior side of a patient torso with a number of EMG electrodes and an accelerometer affixed to the torso, showing the x- and y-axes of the accelerometer defined in relation to the torso, in accordance with an exemplary embodiment of the present invention;

FIG. 1B is a perspective view of the right side of the patient shown in FIG. 1A, showing the x-z plane defined with respect to the torso such that the gravity vector lies approximately in the x-z plane, in accordance with an exemplary embodiment of the present invention;

FIG. 2 shows two example waveforms representative of a raw signal measured by EMG electrodes such as the electrodes shown in FIG. 1A after the raw signal has undergone two different forms of preprocessing, in accordance with exemplary embodiments of the present invention;

FIG. 3 is a flow chart of a method 50 for quantifying respiratory muscle effort, in accordance with exemplary embodiments of the present invention;

FIG. 4 is a flow chart of a process 100 for defining the interval in which a candidate sniff may have occurred during a breathing cycle c of regular breathing preprocessed and sniff preprocessed EMG signals, in accordance with exemplary embodiments of the present invention;

FIG. 5 is a flow chart of a process 200 for defining several features of the main bump of a candidate sniff defined during process 100 using data points and time intervals based on maximum values of the EMG signals determined in process 100, in accordance with exemplary embodiments of the present invention;

FIG. 6 is a flow chart of a process 300 for defining additional features of the main bump of a candidate sniff defined during process 100 using data points and time intervals based on minimum values of the EMG signals determined in process 100, in accordance with exemplary embodiments of the present invention;

FIG. 7 shows a compilation of example waveforms representative of signals measured by EMG electrodes and an accelerometer such as the EMG electrodes and accelerometer depicted in FIGS. 1A and 1B, such example waveforms having undergone various levels of preprocessing according to processes 100-600 depicted in FIGS. 4-6 and 8-10, in accordance with exemplary embodiments of the present invention;

FIG. 8 is a flow chart of a process 400 for constructing a signal that is loosely representative of the change in the angle of the accelerometer shown in FIGS. 1A and 1B as a patient breathes, in accordance with exemplary embodiments of the present invention;

FIG. 9 is a flow chart of a process 500 for constructing upper and lower frequency band signals from a raw accelerometer signal measured by the accelerometer referred to in FIG. 8, in accordance with exemplary embodiments of the present invention; and

FIG. 10 is a flow chart of a process 600 for defining features of the raw accelerometer signal referred to in FIGS. 8 and 9 in order to classify a candidate sniff identified during processes 100-300 as either a sniff or a signal artifact, in accordance with exemplary embodiments of the present invention.

### DETAILED DESCRIPTION OF EXEMPLARY EMBODIMENTS

As used herein, the singular form of “a”, “an”, and “the” include plural references unless the context clearly dictates otherwise.

As used herein, the statement that two or more parts or components are “coupled” shall mean that the parts are joined or operate together either directly or indirectly, i.e., through one or more intermediate parts or components, so long as a link occurs.

As used herein, the term “artifact” shall mean distortions on electromyogram (EMG) signal waveforms due to non-respiratory activity including, but not limited to, activation of non-respiratory muscles (e.g. due to movement of the body, change in electrode-skin impedance, or interference with cables connected to electrodes sensing the EMG signal).

As used herein, the term “controller” shall mean a number of programmable analog and/or digital devices (including an associated memory part or portion) that can store, retrieve, execute and process data (e.g., software routines and/or information used by such routines), including, without limitation, a field programmable gate array (FPGA), a complex programmable logic device (CPLD), a programmable system on a chip (PSOC), an application specific integrated circuit (ASIC), a microprocessor, a microcontroller, a programmable logic controller, or any other suitable processing device or apparatus. The memory portion can be any one or more of a variety of types of internal and/or external storage media such as, without limitation, RAM, ROM, EPROM(s), EEPROM(s), FLASH, and the like that provide a storage register, i.e., a non-transitory machine readable medium, for data and program code storage such as in the fashion of an internal storage area of a computer, and can be volatile memory or nonvolatile memory.

As used herein, the term “machine learning model” shall mean a software system that develops and builds a mathematical model based on sample data, known as “training data”, in order to make predictions or decisions without being explicitly programmed to do so, including, without limitation, a computer software system that develops that has been trained to recognize patterns from a set of training data, and subsequently develops algorithms to recognize patterns from the training data set in other data sets.

As used herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

The present invention, as described in greater detail herein in connection with various particular exemplary embodiments, provides methods and systems for objectively quantifying the breathing effort of a patient, also referred to as respiratory muscle effort (RME), in a non-invasive manner. As described in more detail herein, sniffs are important for accurate determination of RME, but sniffs and signal artifacts often manifest similarly on EMG signal waveforms. While devices such as nasal cannulas or esophageal electrodes can be used to produce additional breathing signals that can be compared to an EMG signal in order to distinguish sniffs from signal artifacts on an EMG waveform, nasal cannulas can be uncomfortable for patients and esophageal electrodes are invasive. The methods and systems of the present invention provide improvements to methods for objectively quantifying RME in two principal ways: by utilizing an accelerometer signal in addition to an EMG signal in order to better distinguish sniffs in an EMG signal waveform from signal artifacts, and by using only non-invasive devices, i.e. EMG electrodes and accelerometers, in order to maximize the comfort of patients while undergoing RME data collection.

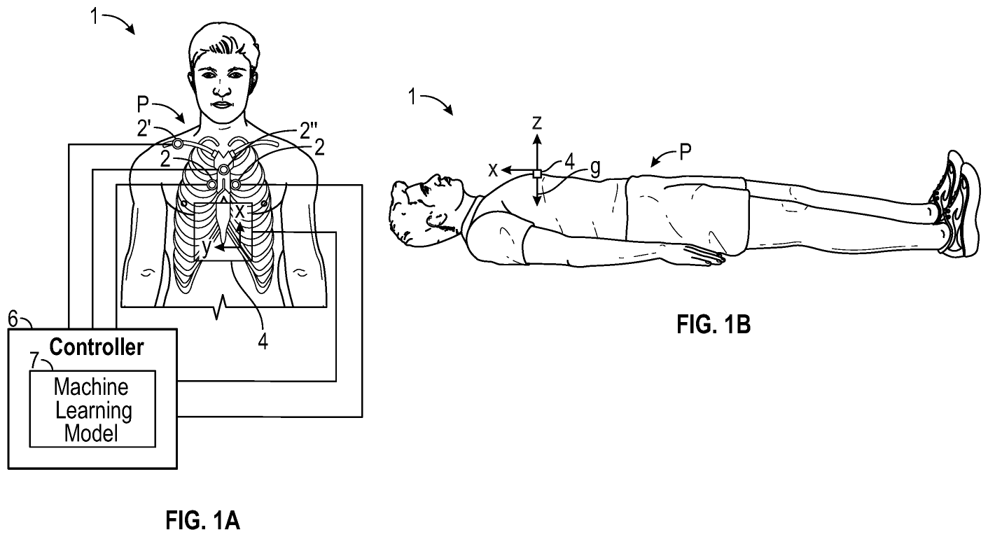

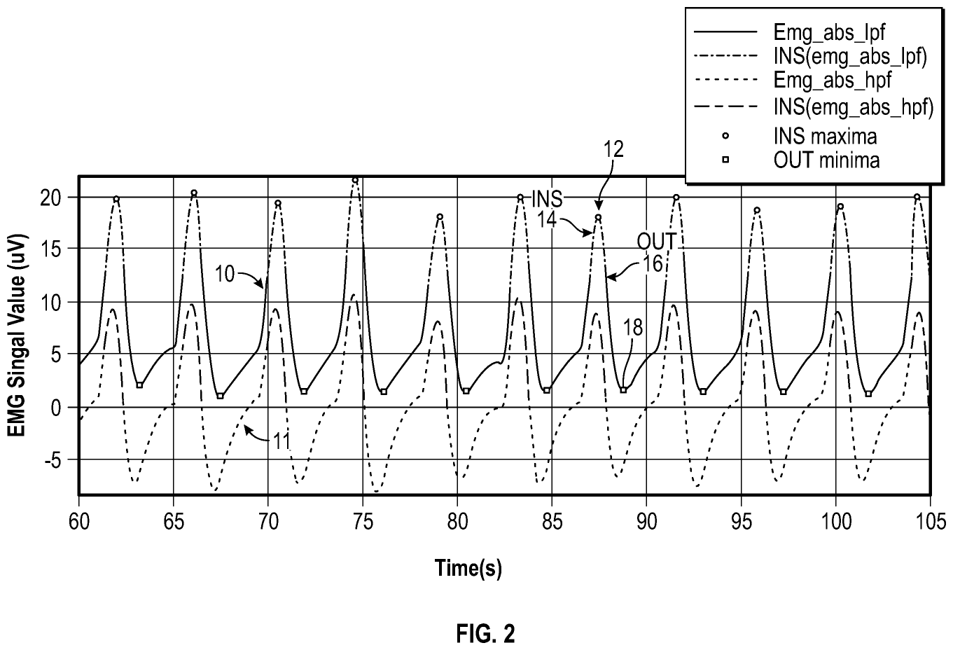

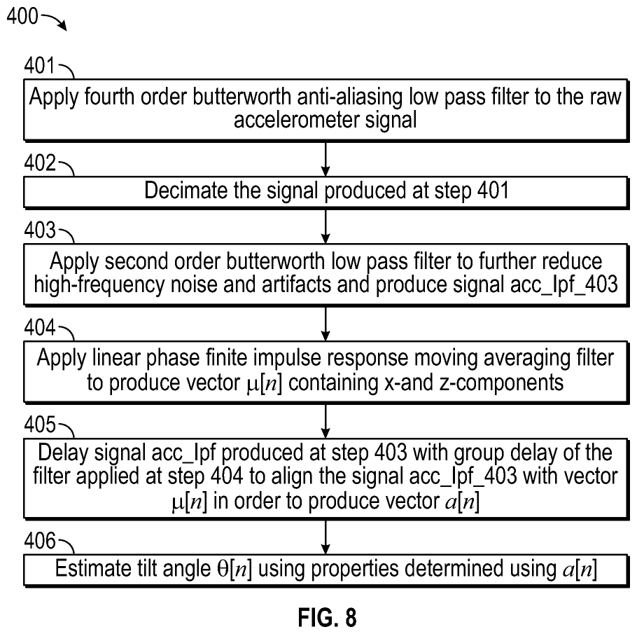

FIGS. 1A and 1B show simplified schematic representations of a respiratory effort quantification system 1 comprised of a number of EMG electrodes 2, a bi- or tri-axial accelerometer 4, and a controller 6. The EMG electrodes 2 are affixed to a patient P in order to monitor respiratory muscle activity of patient P, while accelerometer 4 is affixed to patient P to measure acceleration in a plurality of axes (i.e. the x-, y-, and/or z-axes) of the body surface to which accelerometer 4 is attached (i.e. the thorax) that occurs concurrently with respiratory activity, in accordance with exemplary embodiments of the present invention. Notably, accelerometer 4 also measures accelerations due to artifacts that are entirely unrelated to breathing activity. As explained in more detail herein with respect a process 400 depicted in FIG. 8, the acceleration signals measured by accelerometer 4 are used to construct a signal loosely representative of a change in the orientation of the body surface to which accelerometer 4 is attached. Accordingly, accelerometer 4 can be thought of as either directly or indirectly producing movement and orientation signals.

In an exemplary embodiment, EMG electrodes 2 are placed on the second intercostal space. Placement of a reference electrode can be varied, and FIG. 1A shows two exemplary embodiments of reference electrode placement. In one exemplary embodiment, a reference electrode 2′ is placed on the clavicle, and in another exemplary embodiment, a reference electrode 2″ is placed on the sternum slightly above electrodes 2. Accelerometer 4 is attached to the sternum of patient P such that the z-axis points out of the body of the patient, i.e. is orthogonal to the body surface, while the x- and y-axes lie in the plane tangent to the body surface with the x-axis pointing toward the head of the patient P. Accordingly, the x-z plane is approximately vertical with respect to the earth's surface such that the gravity vector lies approximately in the x-z plane. The EMG electrodes 2 and accelerometer 4 are depicted as being in electrical communication with a controller 6, the controller 6 being configured to receive and store the signals measured by the EMG electrodes 2 and accelerometer 4.

It will be appreciated that several types of controllers, for example various types of computers, are capable of receiving and storing signal information detected by devices such as EMG electrodes 2 and accelerometer 4. Accordingly, any type of controller 6, and any number (i.e. one or more than one) of controllers 6 may be used to receive and store the information transmitted by EMG electrodes 2 and accelerometer 4 without departing from the scope of the disclosed invention. In addition, in an exemplary embodiment, software including a machine learning model 7 is integrated into the controller 6 as shown in FIG. 1 so that the methods and processes of the present invention can be automated.

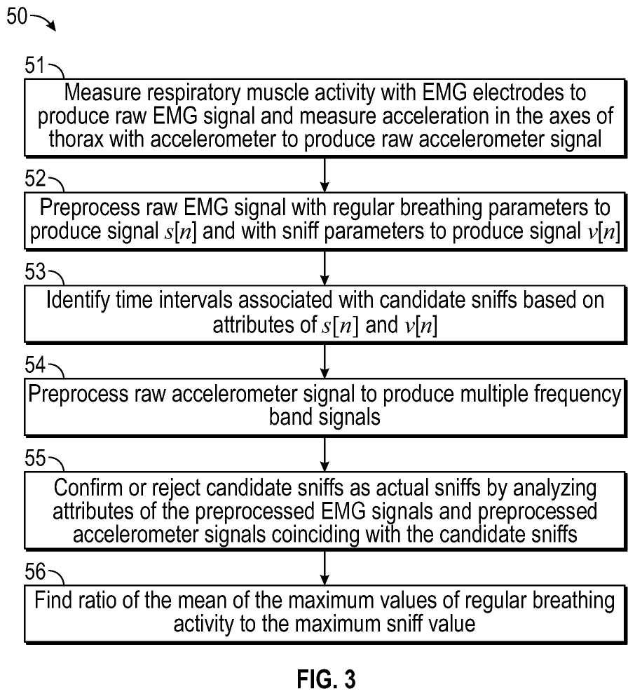

FIG. 2 shows an example of a first preprocessed EMG signal 10 and a second preprocessed EMG signal 11 each depicting multiple breath cycles, with each breath cycle consisting of one significant oscillation in EMG signal 10 or EMG signal 11. EMG signal 10 and EMG signal 11 result from using two different levels of preprocessing on a raw EMG signal measured by electrodes such as EMG electrodes 2 shown in FIG. 1A. Specifically, EMG signal 10 is the result of heavily low-pass filtering a raw EMG signal (i.e. an EMG signal detected by a number of electrodes such as EMG electrodes 2 in FIG. 1A), while EMG signal 11 is the result of performing baseline removal and delay compensation on the EMG signal 10.

EMG signals 10 and 11 in FIG. 2 are shown as preprocessed rather than in raw form in order to better highlight attributes of distinct breath cycles. The methods and systems of the present invention utilize EMG signals that are preprocessed to remove as many signal artifacts as possible while isolating and preserving regular breathing and sniff activity in the signals (the terms “regular breathing” and “sniff” being explained in more detail later herein). While high-level detail about such signal preprocessing is provided herein, the present invention is not directed toward to the granular details of such preprocessing. Hereinafter, references to attributes of the EMG signal will refer to EMG signal 10 for brevity of disclosure, however, it will be appreciated that any discussion of a temporal attribute of EMG signal 10 applies to EMG signal 11 and vice versa, since both signals originate from the same raw EMG signal and span the same interval of time.

EMG signal 10 is segmented into two portions, INS and OUT, as denoted by the use of solid and dashed lines in the waveform in FIG. 2. Those portions of EMG signal 10 containing the maximum of the main peak 12 of a breath cycle are referred to as INS portions 14 of EMG signal 10, and it will be appreciated that all portions of EMG signal 10 that are drawn in the same dashed line as the labeled INS portion 14 also denote INS portions 14 of EMG signal 10. Those portions of EMG signal 10 that are not the INS portions 14 are referred to as the OUT portions 16, and it will be appreciated that all portions of EMG signal 10 that are drawn in the same solid line as the labeled OUT portion 16 also denote OUT portions 16 of EMG signal 10. The INS portion 14 of a given breath cycle c can also be denoted as I[c] and the OUT portion of a given breath cycle c can also be denoted as O[c].

For the methods and systems of the present invention, the exact start and end times of each INS portion 14 and OUT portion 16 do not need to be strictly defined, as the defining feature of a given breath cycle is the inclusion of the maximum of the main peak of a breath cycle in the INS portion. While only one such maximum 12 of a main peak of a breath cycle in EMG signal 10 is labeled in FIG. 2, it will be appreciated that all local maxima occurring during the breath cycles shown in FIG. 2 are also maxima 12 of the main peaks. The time at which a maximum 12 of a main peak of a breath cycle occurs is referred to herein as tₘₐₓ^{rbg}[c]. In viewing FIG. 2, it will be appreciated that each of the OUT portions 16 include the minimum 18 of each breath cycle. While only one such minimum 18 of a breath cycle in EMG signal 10 is labeled in FIG. 2, it will be appreciated that all local minima occurring during the breath cycles shown in FIG. 2 are also minima 18. The time at which a minimum 18 of a breath cycle occurs is referred to herein as tₘᵢₙ^{rbg}[c]. In general, the INS portions 14 of a breath cycle in an EMG signal 10 correspond to inhalations, as respiratory muscle activity is typically largest during inhalations. However, for some patients, respiratory muscle activity is instead greatest during exhalations, and it will be understood that the INS portions 2 of a breath cycle in an EMG signal correspond to exhalations for such patients.

If a given breath cycle is referred to as breath cycle c herein, it should be understood that the breath cycle immediately preceding cycle c is cycle [c−1] and the cycle immediately following cycle c is [c+1]. A breath cycle can be defined as either an algorithmic breath cycle or a natural breath cycle. An algorithmic breath cycle is denoted ABC[c] and comprises the OUT portion 16 of a breath cycle followed by the INS portion 14 immediately following, such that ABC[c]=(O[c], I[c]). A natural breath cycle is denoted as NBC[c] and comprises the INS of a breath cycle followed by the OUT immediately following, such that NBC[c]=(I[c], O[c+1]). The algorithmic breath cycle is so called because defining a breath cycle as (O[c], I[c]) is more conducive to artifact detection by a real-time algorithm than (I[c], O[c+1]).

Prior to proceeding with in-depth descriptions of exemplary embodiments of the present invention, several acronyms and variable names (including the variables discussed above) and their meanings are listed below for ease of reference. Some of the entries in the following list have not yet been introduced, however, the list can be referenced as the acronyms and variables are used throughout the present disclosure:

### List of Acronyms and Variables

i. BC: stands for “breath cycle”, part of an EMG signal consisting of one significant oscillation expected to correspond to either a combination of a successive inhalation and exhalation or a large bump due to an artifact
ii: INS: denotes the portions of a breath cycle containing the maximum of the main peak of the breath cycle
iii. OUT: denotes the portions of the breath cycle that are not INS portions;
iv. I[c]: denotes the INS portion of a breath cycle c
v. O[c]: denotes the OUT portion of a breath cycle c
vi. c: denotes the integer sequential breath cycle index
vii. ABC[c]: stands for “algorithmic breath cycle” which is defined in order to render the artifact detection algorithm used in the present invention meaningful and facilitate real-time processing; each ABC[c] comprises the OUT of a breath cycle followed by the INS immediately following, such that ABC[c]=(O[c], I[c])
viii. NBC[c]: stands for “natural breath cycle”; each NBC [c] comprises the INS of a breath cycle followed by the OUT immediately following, such that NBC[c]=(I[c], O[c+1])
ix. s[n]: denotes an EMG signal preprocessed with regular breathing parameters; the EMG signals 10 and 11 shown in FIG. 2 denote two such EMG signals s[n] preprocessed with regular breathing parameters
x. v[n]: denotes an EMG signal preprocessed with sniff parameters
xi. tₘₐₓ^{rbg}[c]: time associated with maximum value of s[n] during I[c]; referring to FIG. 2, a point 12 represents a local maximum value of a breathing cycle c in s[n], and the time associated with the labeled point 12 is tₘₐₓ^{rbg}[c] for that breathing cycle c
xii. tₘᵢₙ^{rbg}[c]: time associated with minimum value of s[n] during O[c]; referring to FIG. 2, a point 18 represents a local minimum value of a breathing cycle c in s[n], and rbg the time associated with the labeled point 18 is tₘᵢₙ^{rbg}[c] for that breathing cycle c
xiii. tₘᵢₙ^{snf}[c]: time associated with minimum value of v[n] during O[c]
xiv. tₘₐₓ^{snf}[c]: time associated with maximum value of v[n] during I[c]
xv. E_{abs}^{rbg}: denotes the mean of the maxima of absolute EMG activity of breath cycles that are deemed artifact-free by artifact detection for regular breathing
xvi. E_{abs}^{snf}: denotes the maximum respiratory muscle activity measured during sniffs after sniffs are identified in an EMG signal using features of an accelerometer signal simultaneously with features of the EMG signal
xvii. Eᵣₑₗ: denotes the ratio of E_{abs}^{rbg} to E_{abs}^{snf} and constitutes a relative measurement of respiratory muscle effort (RME)

As previously stated, the methods and systems of the present invention utilize EMG signals that are preprocessed to reduce signal artifacts as much as possible, which allows regular breathing and sniff activity from a raw EMG signal to be isolated. “Regular” breathing is considered to occur when the patient is assumed to be relaxed and breathing naturally or spontaneously. In comparison, a “sniff” is much sharper, stronger, and shorter than a regular breath. It should be noted that the unique nature of sniffs (i.e. the sharp, strong, and short nature as compared to regular breathing inhalations) and their relatively infrequent occurrence compared to regular breaths, does not immediately lend to the natural detection of sniffs by algorithms designed to detect breath cycles and artifacts during regular breathing activity.

Hence, a set of regular breathing parameters designed to detect regular breathing activity is used to preprocess a given raw EMG signal to produce a regular breathing signal s[n] (such preprocessing may be referred to hereinafter as “regular breathing preprocessing”) such as EMG signals 10 and 11 in FIG. 2, and a different set of sniff parameters designed to detect sniff activity and preserve the main characteristics of sniffs is used to preprocess a given EMG signal to produce a sniff signal v[n] (such preprocessing may be referred to hereinafter as “sniff preprocessing”). As such, in accordance with exemplary embodiments of the present invention, for a given raw EMG signal, both a regular breathing preprocessed signal s[n] and a sniff preprocessed signal v[n] are produced such that, for each point in time represented by a data point in the signal s[n], there is a corresponding data point in the signal v[n] and vice versa.

Preprocessing of an EMG signal to produce a regular breathing signal s[n] and a sniff preprocessed signal v[n] follow several of the same steps, with the primary distinction between regular breathing preprocessing and sniff preprocessing being that sniff preprocessing involves much less net smoothing of the EMG signal. Both regular breathing preprocessing and sniff preprocessing involve spike removal, scaling, a first round of high-pass filtering, optional powerline interference reduction, rectification, downsampling, median filtering, and slight low-pass filtering. Regular breathing processing additionally includes another round of low pass filtering, another round of high pass filtering for baseline removal, and construction of auxiliary signals for breath cycle part and artifact detection.

Spike removal eliminates very short large spikes in the raw EMG signal due to, for example and without limitation, pacemakers and sharp artifacts. Scaling simply refers to converting a signal measured in volts to units of microvolts (or another convenient unit). The first round of high-pass filtering reduces low-frequency motion artifacts, tonic activity, electrocardiogram (ECG) activity, powerline interference, and sensor noise. If residual powerline interference is present after the first round of high-pass filtering, a comb filter with notches at the fundamental powerline frequency and its harmonics can be used to further reduce the powerline interference. Rectification is the first operation for computing a low-frequency envelope of the high-frequency EMG signal, in order to construct a surrogate respiration signal. Downsampling follows rectification to reduce memory and processing requirements, since the frequencies present in the envelope signal are much lower than the frequencies in the raw signal. Median filtering is applied to further reduce remaining spikes, in particular due to ECG. Slight low-pass filtering is used to further smooth the envelope signal and obtain a surrogate respiratory muscle activity signal. The further smoothing from slight low-pass filtering facilitates computation of minima and maxima in the preprocessed EMG signal.

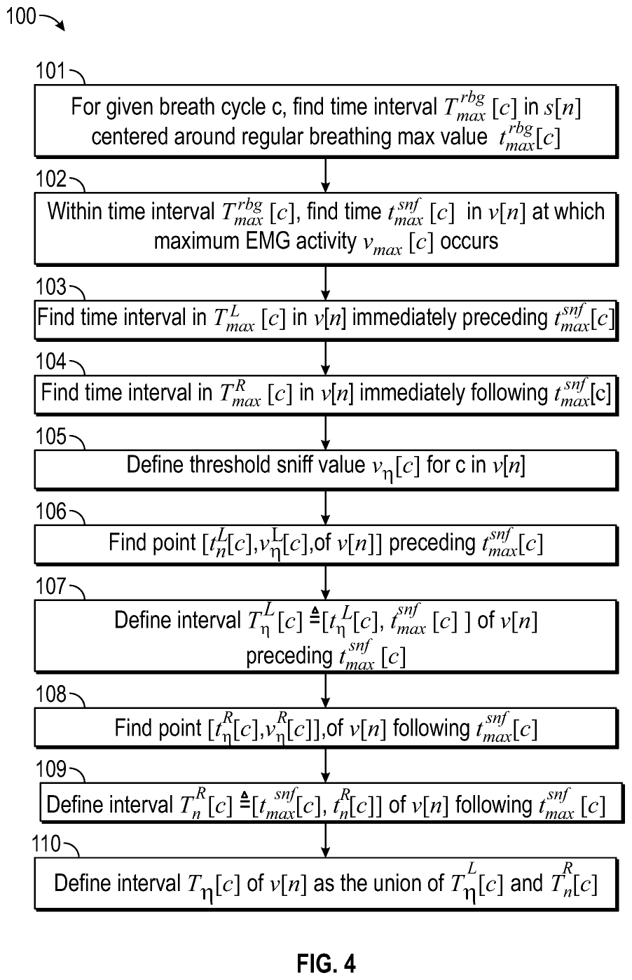

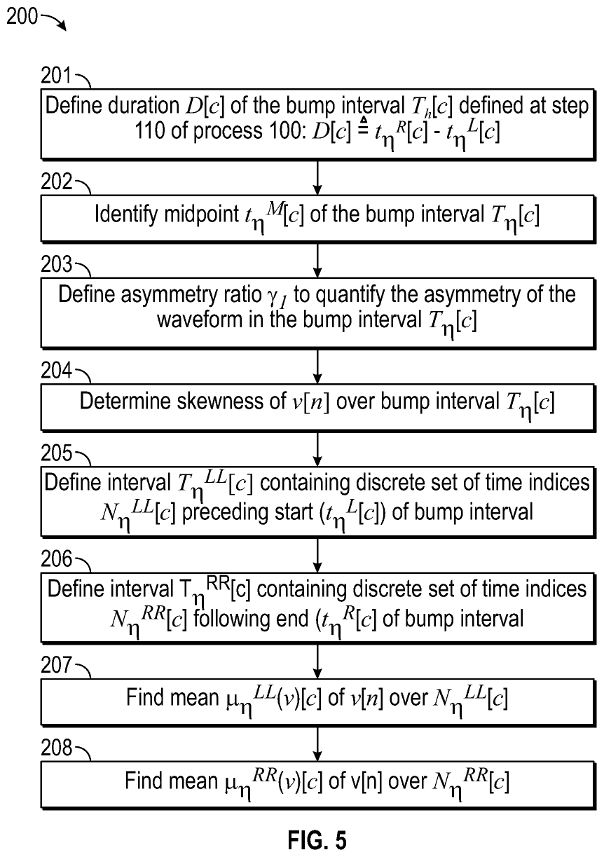

Referring now to FIG. 3, a flow chart depicting a method 50 for quantifying RME is shown. It should be noted that the steps of method 50 provide a general overview of the steps involved in quantifying RME, and that most of the steps of method 50 are themselves processes that involve several steps. Accordingly, more specific details of each of the steps of method 50 are provided herein with respect to the processes 100, 200, 300, 400, 500, and 600 depicted in FIGS. 4-6 and 8-9. At step 51 of method 50 and as previously described with respect to FIGS. 1A and 1B, respiratory muscle activity is measured with EMG electrodes 2 and acceleration in the x-, y-, and/or z-axes of the thorax concurrent with respiratory activity is measured with accelerometer 4 in order to produce raw EMG and accelerometer signals. At step 52, the raw EMG signal measured by EMG electrodes 2 is preprocessed with regular breathing parameters to produce a regular breathing signal s[n] and with sniff parameters to produce a sniff signal v[n]. Throughout the present disclosure, a portion of a preprocessed EMG signal s[n] or v[n] that represents a potential sniff may be referred to as a “candidate sniff”. At step 53, various attributes of regular breathing signal s[n] and sniff signal v[n] are used to identify time intervals where candidate sniffs have occurred. More details regarding steps 51-53 of method 50 are provided herein with respect to a process 100 depicted in FIG. 4.

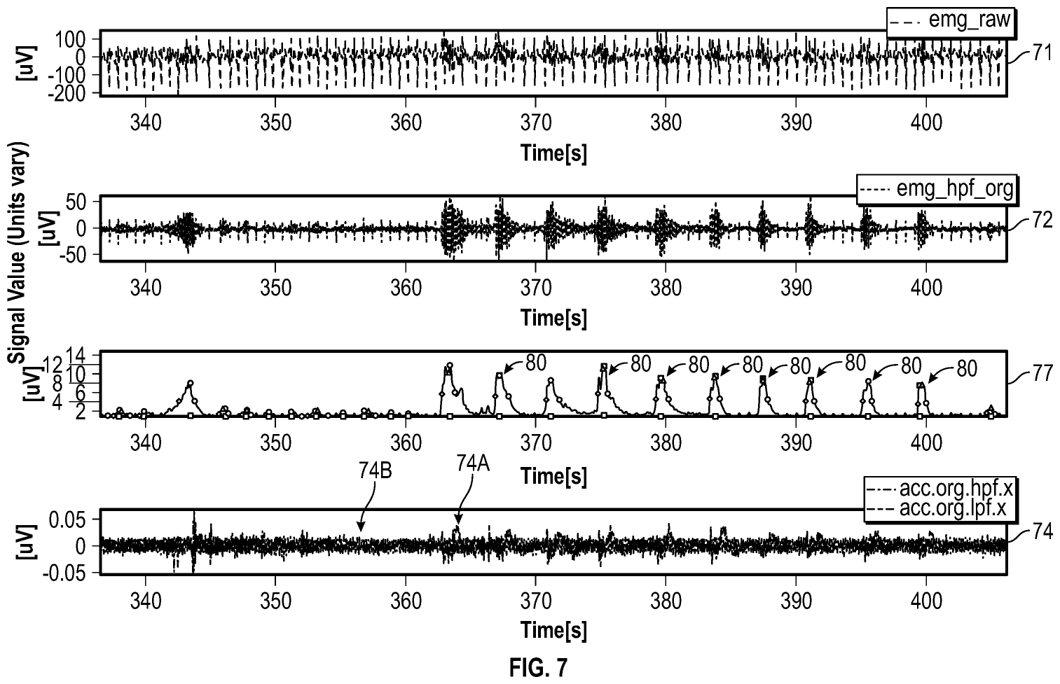

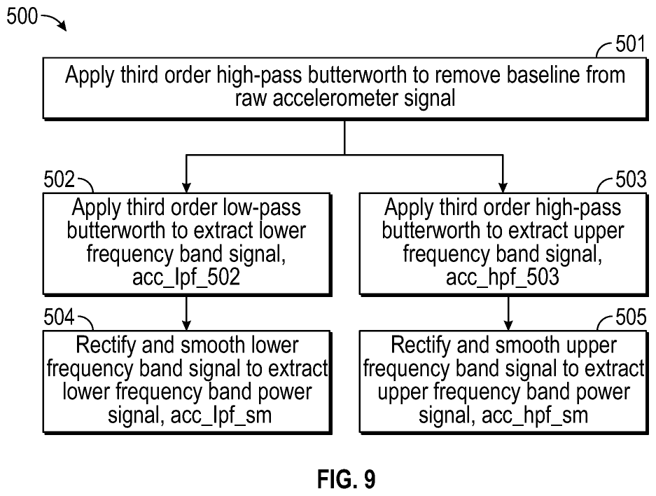

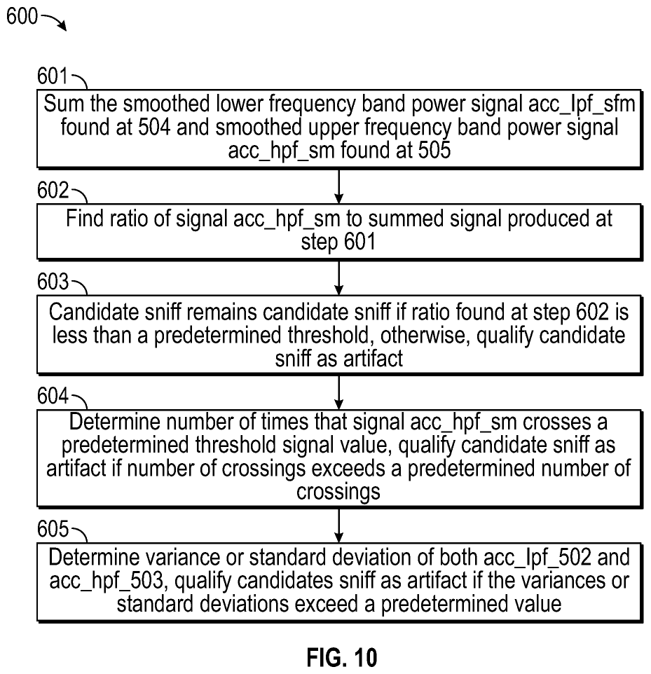

Still referring to FIG. 3, at step 54 of method 50, the raw accelerometer signals measured by accelerometer 4 are preprocessed to produce multiple signals at distinct frequency bands. More details regarding step 54 are provided herein with respect to process 500 depicted in FIG. 9. At step 55, attributes of the preprocessed EMG signals and the preprocessed accelerometer signals found at steps 52-54 are used to confirm or reject candidate sniffs as actual sniffs by analyzing attributes of the preprocessed accelerometer signals coinciding with the candidate sniffs. More details regarding step 55 of method 50 are provided herein with respect to process 300 and 600 depicted in FIGS. 6 and 10, respectively.

The final step 56 of method 50 is the final step in quantifying RME, which requires using the regular breathing attribute E_{abs}^{rbg} determined during process 100 depicted in FIG. 4 (which corresponds to steps 51-53 of method 50), the sniff attribute E_{abs}^{snf} determined by executing steps 51-55 of method 50 and the corresponding processes 100-600 depicted in FIGS. 4-6 and 8-10, and using E_{abs}^{rbg} and E_{abs}^{snf} to determine a value Eᵣₑₗ. E_{abs}^{rbg} represents the mean of the maximum values of regular breathing activity that are deemed artifact-free, while E_{abs}^{snf} denotes the maximum value of detected sniff activity. In step 56 of method 50, the attribute Eᵣₑₗ is determined by normalizing the regular breathing value E_{abs}^{rbg} with respect to the sniff value E_{abs}^{snf} as follows:

$${E}_{rel} = \frac{{E}_{abs}^{rbg}}{{E}_{abs}^{snf}}$$

such that E_{abs}^{rbg} can be expressed as a fraction or percentage of E_{abs}^{snf}. Step 56 of method abs 50 is performed because experimental results show that Eᵣₑₗ is one of the most predictive measures of RME.

FIGS. 4-6 and 8-10 are flow charts detailing the individual steps of various processes 100-600 that are used to extract regular breathing and sniff characteristics from EMG signals using both the EMG signals and accelerometer signals. Processes 100-600 are intended to be performed for every breath cycle c in a given signal waveform and were developed using a classical feature extraction approach. It should be noted that, for example and without limitation, when reference is made to finding the minimum or maximum value of a signal waveform within a given breath cycle c, said breath cycle c and said minimum or maximum value of the signal waveform within the breath cycle c are identified by the trained machine learning model 7 and checked for correctness manually. Accordingly, it should also be noted that the EMG and accelerometer waveforms that are referred to in the detailed descriptions of processes 100-600 can contain either or both of sniffs performed by the patient on command or performed spontaneously, as long as the practitioner can note the timing of such sniff and use the knowledge of the timing to verify the efficacy of processes 100-600.

FIG. 4 is a flow chart detailing the steps of a process 100 for defining the interval in which a candidate sniff may have occurred during a given breathing cycle c of regular breathing preprocessed EMG signal s[n] and the corresponding sniff preprocessed EMG signal v[n], in accordance with exemplary embodiments of the present invention. As previously stated, the time associated with the maximum value of s[n] during I[c] is tₘₐₓ^{rbg}[c], and the time associated with the minimum value of s[n] during O[c] is tₘᵢₙ^{rbg}[c]. In process 100, the time tₘₐₓ^{rbg}[c] is used as an initial seed point in each breath cycle c to identify several fiducial points in v[n]. The time tₘᵢₙ^{rbg}[c] is similarly used as an initial seed point to identify additional fiducial points in v[n], as described later herein with respect to a process 300 depicted in FIG. 6

At step 101 of process 100, a time interval Tₘₐₓ^{rbg}[c] centered around the time tₘₐₓ^{rbg}[c] is defined as follows:

$${T}_{max}^{rbg} [ c ] {=}^{Δ} [ {t}_{max}^{rbg} [ c ] - {T}_{max ,} , {t}_{max}^{rbg} [ c ] + {T}_{max} ]$$

with Tₘₐₓ being a pre-specified duration which, in an exemplary embodiment, is typically set to 0.8 seconds. Hence Tₘₐₓ^{rbg}[c] is the interval of duration 2Tₘₐₓ centered at the time tₘₐₓ^{rbg}[c] associated with the maximum value of I[c] in s[n]. At step 102, within the time interval Tₘₐₓ^{rbg}[c] defined at step 101, the time tₘₐₓ^{snf}[c] in signal v[n] at which maximum EMG activity vₘₐₓ[c] occurs is identified. At step 103, a time interval Tₘₐₓ^{L}[c] to the left of (i.e. immediately preceding) tₘₐₓ^{snf}[c] in v[n] is defined as follows:

$${T}_{max}^{L} [ c ] {=}^{Δ} [ {t}_{max}^{snf} [ c ] - {T}_{max}^{L} , {t}_{max}^{snf} [ c ] ]$$

with Tₘₐₓ^{L} being a pre-specified value which, in an exemplary embodiment, is typically set to be 1.2 seconds. Similarly, at step 104, a time interval Tₘₐₓ^{R}[c] to the right of (i.e. immediately following) tₘₐₓ^{snf}[c] in v[n] is defined as follows:

$${T}_{max}^{R} [ c ] {=}^{Δ} [ {t}_{max}^{snf} [ c ] , {t}_{max}^{snf} [ c ] + {T}_{max}^{R} ]$$

with Tₘₐₓ^{R} being a pre-specified value which, in an exemplary embodiment, is typically set to be 1.6 seconds.

At step 105, a threshold sniff value v_{η}[c] for breathing cycle c in the sniff signal v[n] is defined as follows:

$${ν}_{η} [ c ] {=}^{Δ} η {ν}_{max} [ c ]$$

where η is a pre-specified sniff coefficient which, in an exemplary embodiment, is typically set to be 0.5, such that the threshold sniff value v_{η}[c] for breathing cycle c in sniff signal v[n] is equal to 0.5 times the maximum value vₘₐₓ[c] of v[n] for that particular breathing cycle c. At step 106, the last point [tₙ^{L}[c], v_{η}^{L}[c]] for which tₙ^{L}[c]∈Tₘₐₓ^{L}[c] and v_{η}^{L}[c]<v_{η}[c] is identified. This point [tₙ^{L}[c], v_{η}[c]] is the last point in time to the left of (i.e. before) the maximum tₘₐₓ^{snf}[c] that is at most Tₘₐₓ seconds away from tₘₐₓ^{snf}[c], and where the value of v[n] drops below the threshold value v_{η}[c] for the first time when looking back from the maximum. At step 107, a left time interval T_{η}^{L}[c] is defined using the time t_{η}^{L}[c] found at step 106 as follows:

$${T}_{η}^{L} [ c ] {=}^{Δ} [ {t}_{η}^{L} [ c ] , {t}_{max}^{snf} [ c ] ]$$

Similarly, at step 108, the first point [t_{η}^{R}[c], v_{η}^{R}[c]] for which t_{η}^{R}[c]∈Tₘₐₓ^{R}[c] and v_{η}^{R}[c]<v_{η}[c] is identified. This point is the first point in time to the right of (i.e. after) the maximum tₘₐₓ^{snf}[c] that is at most Tₘₐₓ^{R} seconds away from tₘₐₓ^{snf}[c], and where the value of v[n] drops below the threshold value v_{η}[c] for the first time when looking forward from the maximum. At step 109, a right time interval Tₙ^{R}[c] is defined using the time t_{η}^{R}[c] found at step 108 as follows:

$${T}_{n}^{R} [ c ] {=}^{Δ} [ {t}_{max}^{snf} [ c ] , {t}_{n}^{R} [ c ] ]$$

At the last step 110, an interval defined by the threshold sniff value v_{η}[c] and lying around the time tₘₐₓ^{snf}[c] is defined as follows:

$${T}_{η} [ c ] {=}^{Δ} {T}_{η}^{L} [ c ] ⋃ {T}_{n}^{R} [ c ] = [ {t}_{η}^{L} [ c ] , {t}_{η}^{R} [ c ] ]$$

The interval T_{η}[c] defines the main part of a candidate sniff in relation to the threshold sniff value v_{η}[c], and the interval T_{η}[c] may be referred to hereinafter as a “bump” or “bump interval” in the preprocessed EMG signal v[n]. It will be appreciated that the boundaries of the interval T_{η}[c] comprise the left boundary of the time interval T_{η}^{L}[c] identified at step 106 and the right boundary of the time interval Tₙ^{R}[c] identified at step 108.

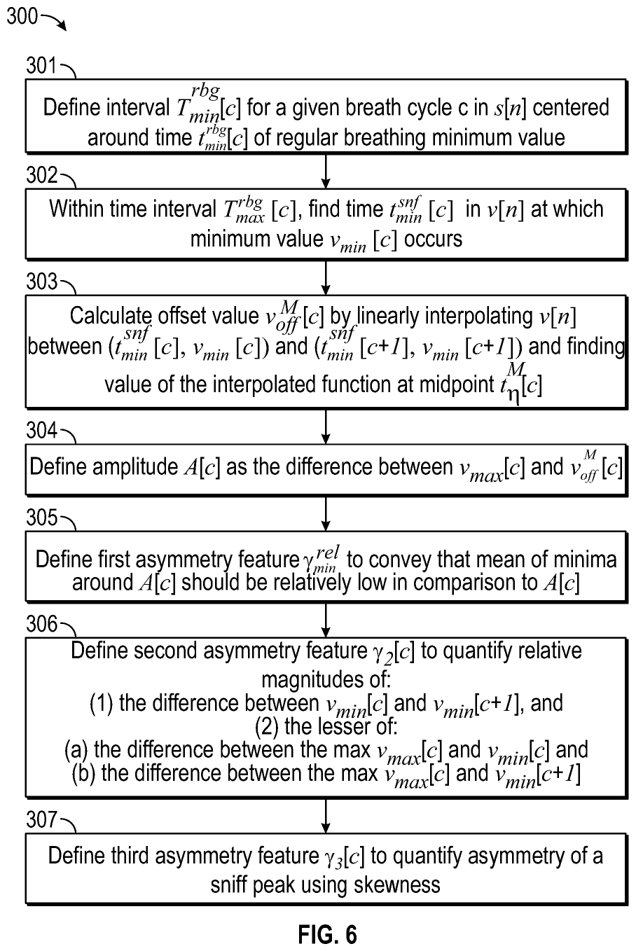

FIG. 5 is a flow chart detailing the steps of a process 200 for defining several features of the main bump of a candidate sniff using the preprocessed EMG signals s[n] and v[n] and the data points and time intervals determined in process 100. At step 201, the duration D[c] of the bump interval T_{η}[c] defined at step 110 of process 100 is defined as follows:

$$D [ c ] {=}^{Δ} {t}_{η}^{R} [ c ] - {t}_{η}^{L} [ c ]$$

This duration D[c] is required to lie between a minimum value Dₘᵢₙ and a maximum value Dₘₐₓ. In an exemplary embodiment of the present invention, Dₘᵢₙ is typically chosen to be 0.15 seconds in and Dₘₐₓ is typically chosen to be 1.0 seconds. At step 202, a midpoint t_{η}^{M}[c] of the bump interval T_{η}[c] is identified such that one half of the area under the curve of v[n] in the bump interval T_{η}[c] lies between t_{η}^{L}[c] and t_{η}^{M}[c] (i.e. to the left of t_{η}^{M}[c]), and the second half lies between t_{η}^{M}[c] and t_{η}^{R}[c] (i.e. to the right of t_{η}^{M}[c]). Accordingly, the midpoint t_{η}^{M}[c] can also be referred to as the median of the bump interval T_{η}[c]. The midpoint t_{η}^{M}[c] is identified because it represents a better “center of mass” than tₘₐₓ^{snf}[c], as vₘₐₓ[c] may be more sensitive to small local extrema that may be present in the bump interval T_{η}[c]. At step 203, an asymmetry ratio γ₁ is defined to quantify the asymmetry of the waveform in the bump interval T_{η}[c]:

$${γ}_{1} = \frac{{t}_{η}^{R} [ c ] - {t}_{η}^{M} [ c ]}{{t}_{η}^{M} [ c ] - {t}_{η}^{L} [ c ]}$$

As one may intuit, empirical data shows that sniffs typically rise faster than they decay. Therefore, the asymmetry ratio would typically ideally be greater than 1 and not significantly less than 0.8. In an exemplary embodiment of the present invention, a typical threshold chosen to be the minimum acceptable value of γ₁ is 0.85.

At step 204, the skewness of v[n] between t_{η}^{L}[c] and t_{η}^{R}[c] (i.e. over the bump interval T_{η}[c]) is determined, in order to have a second measure of the asymmetry of the waveform in the bump interval T_{η}[c]. Again, because sniffs typically rise faster than they decay, it is expected that the skewness of the bump interval T_{η}[c] would be positive (i.e. skewed to the right). In an exemplary embodiment of the present invention, a typical threshold for the skewness over the bump interval T_{η}[c] is zero.

Intuitively, sniffs performed properly cannot occur too closely together. Therefore, large bumps in v[n] that occur just before or just after T_{η}[c] are very likely due to artifacts and can impact the candidate sniff. To detect such bumps, at steps 205 and 206, an interval T_{η}^{LL}[c] to the left of t_{η}^{L}[c] (i.e. an interval preceding the beginning of the bump interval T_{η}[c]) and an interval T_{η}^{RR}[c] to the right of t_{η}^{R}[c] (i.e. an interval following the end of the bump interval T_{η}[c]), are defined as follows:

$${T}_{η}^{LL} [ c ] {=}^{Δ} [ {t}_{η}^{L} [ c ] - {T}_{bump} , {t}_{η}^{L} [ c ] ]$$

$${T}_{η}^{RR} [ c ] {=}^{Δ} [ {t}_{η}^{R} [ c ] , {t}_{η}^{R} [ c ] + {T}_{bump} ]$$

where T_{bump} is a pre-specified duration which, in an exemplary embodiment, is set to a typical value of 0.5 s. Corresponding sets of discrete time indices within each interval T_{η}^{LL}[c] and T_{η}^{RR}[c] are denoted as N_{η}^{LL}[c] and N_{η}^{RR}[c], respectively. At steps 207 and 208, the values μ_{η}^{LL}(v)[c] and μ_{η}^{RR}(v)[c] representing the mean of v[n] over N_{η}^{LL}[c] and the mean of v[n] over N_{η}^{RR}[c], respectively, are calculated, with μ_{η}^{LL}(v)[c] and μ_{η}^{RR}(v)[c] being defined as follows:

$${μ}_{η}^{LL} ( v ) [ c ] {=}^{Δ} mean ( \{ v [ n ] \} , n ∈ {N}_{η}^{LL} [ c ] )$$

$${μ}_{η}^{RR} ( v ) [ c ] {=}^{Δ} mean ( \{ v [ n ] \} , n ∈ {N}_{η}^{RR} [ c ] )$$

Both means μ_{η}^{LL}(v)[c] and μ_{η}^{RR}(v)[c] are required to be less than the threshold value v_{η}[c] calculated at step 105 of process 100 such that μ_{η}^{LL}(v)[c]<v_{η}[c] and μ_{η}^{RR}(v)[c]<v_{η}[c]. In the absence of undesired bumps that occur close to a potential sniff (bumps being “undesired” as they complicate the isolation and identification of a sniff), the values μ_{η}^{LL}(v)[c] and μ_{η}^{RR}(v)[c] can be used as additional figures for quantifying the asymmetry of the candidate sniff. Again, because sniffs are expected to rise faster than they decay, the following criterion is imposed:

$${μ}_{η}^{LL} ( v ) [ c ] < {μ}_{η}^{RR} ( v ) [ c ]$$

Processes 100 and 200 depicted in FIGS. 4 and 5 use time stamps and values related to fiducial points in s[n] and v[n] derived from the maxima of the EMG signal. However, the minima of an EMG signal are also highly relevant to the quantification of RME. Accordingly, FIG. 6 is a flow chart detailing the steps of a process 300 for defining additional features of a candidate sniff based on the minima of preprocessed EMG signals s[n] and v[n] using data points and time intervals determined in processes 100 and 200. Specifically, process 300 is used to determine EMG-derived features and attributes that can be used to differentiate between sniff and non-sniff (e.g. artifact) activity. As previously stated, the time associated with the minimum value of s[n] during O[c] is tₘᵢₙ^{rbg}[c], and in process 300, time tₘᵢₙ^{rbg}[c] is used as an initial seed point in each breath cycle c to identify several fiducial points in v[n].

At step 301 of process 300, a time interval Tₘᵢₙ^{rbg}[c] in s[n] for a given breath cycle c is defined as follows:

$${T}_{min}^{rbg} [ c ] {=}^{Δ} [ {t}_{min}^{rbg} [ c ] - {T}_{min ,} , {t}_{min}^{rbg} [ c ] + {T}_{min} ]$$

with Tₘᵢₙ being a pre-specified duration which, in an exemplary embodiment, is typically set to 0.8 s. Hence, Tₘᵢₙ^{rbg}[c] is the interval in s[n] of duration 2Tₘᵢₙ centered at the time tₘᵢₙ^{rbg}[c] associated with the minimum value of O[c]. At step 302, within the time interval Tₘᵢₙ^{rbg}[c] defined at step 301, the time tₘᵢₙ^{snf}[c] in signal v[n] at which the minimum value vₘᵢₙ[c] occurs is identified. At step 303, an offset value v_{offs}^{M}[c] of the c-th breath cycle is found by first linearly interpolating v[n] between the minimum (tₘᵢₙ^{snf}[c], vₘᵢₙ[c]) of the c-th breath cycle and the minimum (tₘᵢₙ^{snf}[c+1], vₘᵢₙ[c+1]) of the (c+1)-th breath cycle, then finding the value of the interpolated function at the location of the midpoint t_{η}^{M}[c]. Subsequently, at step 304, an amplitude of the c-th breath cycle is defined as the difference between vₘₐₓ[c] and v_{offs}^{M}[c]:

$$A [ c ] {=}^{Δ} {v}_{max} [ c ] - {v}_{offs}^{M} [ c ]$$

where the criteria A^{low}≤A[c]≤A^{upp} is imposed on the amplitudes. In an exemplary embodiment, typical values for the lower and upper thresholds A^{low} and A^{upp} are 3 μV and 40 μV, respectively.

At step 305, a first asymmetry feature γₘᵢₙ^{rel} is defined in order to quantify the fact that it is preferable for the mean of the minima preceding and following the maximum (tₘₐₓ[c], vₘₐₓ[c]) to be low in value relative to the amplitude A[c] of the corresponding breath cycle. As such, γₘᵢₙ^{rel} is defined as follows:

$${γ}_{min}^{rel} {=}^{Δ} \frac{{v}_{min} [ c ] + {v}_{min} [ c + 1 ]}{2 A [ c ]}$$

where γₘᵢₙ^{rel} is required to be less than a pre-specified threshold which, in an exemplary embodiment, is chosen to be approximately 0.3. At step 306, a second asymmetry feature γ₂[c] is defined to quantify the relative magnitudes of the difference between subsequent valley minima vₘᵢₙ[c] and vₘᵢₙ[c+1] on the one hand, and the lesser of the differences between the maximum of the peak vₘₐₓ[c] in between the valley minima and each of the valley minima (vₘᵢₙ[c] and vₘᵢₙ[c+1]) on the other hand. As such, γ₂[c] is defined as follows:

$${γ}_{2} [ c ] {=}^{Δ} \frac{\vert {v}_{min} [ c + 1 ] - {v}_{min} [ c ] \vert}{min ( \vert {v}_{max} [ c ] - {v}_{min} [ c ] \vert , \vert {v}_{max} [ c ] - {v}_{min} [ c + 1 ] \vert )}$$

where γₘᵢₙ^{rel} is required to be less than a pre-specified upper threshold which, in an exemplary embodiment, is chosen to be approximately 0.2. Lastly, at step 307, a third asymmetry feature γ₃[c] is defined to quantify the left asymmetry of the sniff peak as follows:

$${γ}_{3} [ c ] {=}^{Δ} \frac{{v}_{min} [ c ]}{{v}_{max} [ c ]}$$

where γ₃[c] is required to be less than a pre-specified upper threshold which, in an exemplary embodiment, is chosen to be approximately 0.25. In an exemplary embodiment of the disclosed concept, if the candidate sniff does not meet all of the amplitude and asymmetry conditions described above with respect to steps 304-307, the candidate sniff is classified as an artifact rather than a sniff.

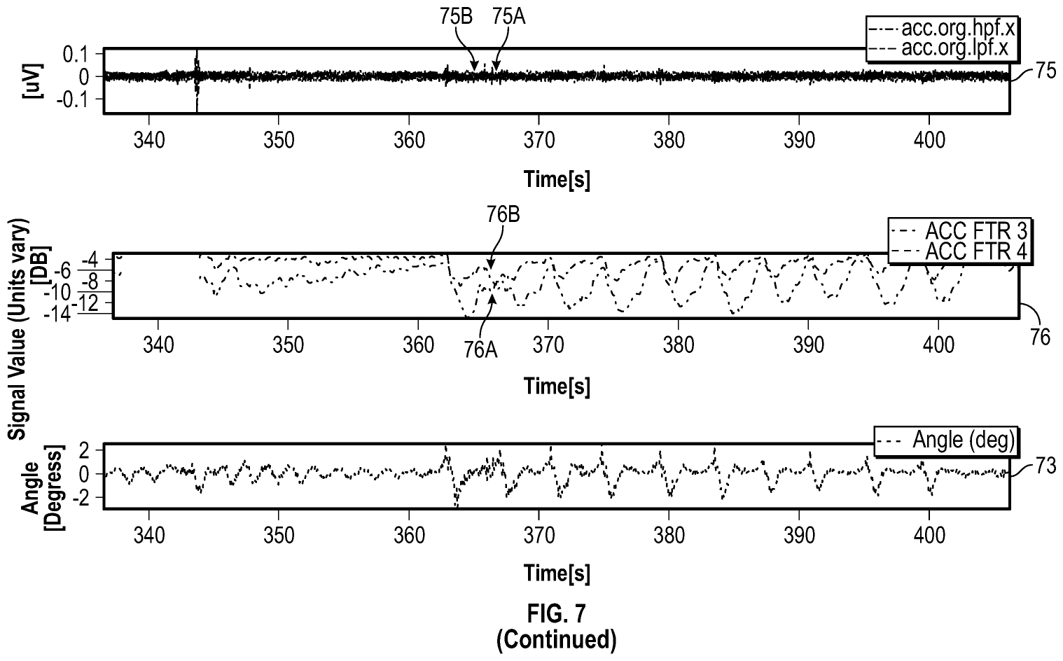

FIG. 7 shows a compilation of a number of waveforms 71, 72, and 77 of an EMG signal that has undergone various levels of preprocessing aligned with a number of waveforms 73, 74, 75, and 76 of an accelerometer signal that has undergone various levels of preprocessing, the EMG signal and accelerometer signal having been measured contemporaneously as depicted by EMG electrodes 2 and accelerometer 4 in FIGS. 1A and 1B, in accordance with exemplary embodiments of the disclosed concept. The waveforms shown in FIG. 7 are used for illustrative purposes only and are not intended to be limiting on the scope of the present invention. The waveforms 71-77 depict raw or processed EMG or accelerometer signals from the same interval of time, but each waveform accentuates a different aspect of respiratory muscle activity or changes in body surface orientation.

Still referring to FIG. 7, waveform 71 depicts a raw EMG signal, and waveform 72 depicts waveform 71 after having undergone preprocessing high-pass filtering. In comparing waveform 72 to waveform 71, it can be appreciated that preprocessing of a raw EMG signal accentuates respiratory muscle activity while decreasing signal artifact. As described herein with respect to process 400 depicted in FIG. 8, waveform 73 depicts a preprocessed accelerometer signal constructed from a raw accelerometer signal that is loosely representative of the change in angle of accelerometer 4 over time, whether the change in angle is due to respiratory activity or non-respiratory activity. Waveforms 74 are preprocessed accelerometer signals (constructed from the raw accelerometer signal) that depict both lower and upper frequency band signals (waveforms 74A and 74B, respectively, in FIG. 7) of the x-axis of accelerometer 4, and waveforms 75 are preprocessed accelerometer signals (also constructed from the raw accelerometer signal) that depict both lower and upper frequency band signals (75A and 75B, respectively, in FIG. 7) of the z-axis of accelerometer 4. The construction of waveforms 74, 75 is described herein with respect to process 600 depicted in FIG. 10.

Still referring to FIG. 7, waveforms 76 represent ratios of upper and lower frequency band signals for both the x-channel and the z-channel of accelerometer 4. Specifically, as described herein with respect to process 500 depicted in FIG. 9, a rectified and smoothed lower frequency band signal acc_lpf_sm is found at step 504 of process 500 (acc_lpf_sm_x is found for the x-channel, and acc_lpf_sm_z is found for the z-channel), and a rectified and smoothed upper frequency band signal acc_hpf_sm is found at step 505 of process 500 (acc_hpf_sm_x is found for the x-channel, and acc_hpf_sm_z is found for the z-channel). In FIG. 7, waveform 76A is the ratio (acc_hpf_sm_x)/(acc_lpf_sm_x+acc_hpf_sm_x) expressed in decibels and waveform 76B is the ratio (acc_hpf_sm_z)/(acc_lpf_sm_z+acc_hpf_sm_z) expressed in decibels. Lastly, waveform 77 represents a sniff processed EMG signal v[n] in which a number of sniffs 80 have been correctly identified after method 50 and the steps of processes 100-600 have been executed for each of the breath cycles identified in the preprocessed EMG signals.

Referring briefly back to FIGS. 1A and 1B and as previously stated, accelerometer 4 is attached to the sternum of the patient P such that the z-axis points out of the body of the patient P and is orthogonal to the body surface, while the x- and y-axes lie in the plane tangent to the body surface with the x-axis pointing toward the head of the patient P. As such, the x-z plane is approximately vertical with respect to the earth's surface such that the gravity vector lies approximately in the x-z plane. Referring now to FIG. 8, a process 400 is used to construct an accelerometer signal that is loosely representative of the change in angle of accelerometer 4 over time, such change in angle being due to some combination of respiratory activity and non-respiratory activity. At a first step 401, a fourth order Butterworth anti-aliasing low pass filter is applied to the raw accelerometer signal, and in an exemplary embodiment, a cutoff frequency of 20 Hz is used. At step 402, the resulting signal is decimated. In an exemplary embodiment of the present invention, the signal is decimated by a factor of 16. At step 403, a second order Butterworth low pass filter is applied to further reduce high-frequency noise and artifacts of the accelerometer signal, and the resulting signal is referred to hereinafter as acc_lpf_403. In an exemplary embodiment, a cutoff frequency of 10 Hz is applied.

At step 404, a linear phase finite impulse response moving averaging filter is applied. In an exemplary embodiment of the present invention, said linear phase finite impulse response moving averaging filter is applied with a support of 2 seconds. Step 404 produces a vector μ[n] containing x- and z-components as the first and second elements, respectively, of the vector. At step 405, the signal acc_lpf_403 produced at step 403 is delayed with the group delay of the filter in order to align it with vector u[n], producing a vector a[n]. At step 406, a tilt angle θ[n] that is loosely representative of the angle resulting from changes in the orientation of the body surface (i.e. the x-y plane as depicted in FIG. 1A) to which the accelerometer 4 is attached is estimated using the equation below, by finding the y-component of the cross-product of μ[n] and a[n] (i.e. the sine of the angle between u[n] and a[n]) based on the previously-established assumption that the gravity vector lies approximately in the x-z plane and normalizing the cross-product by the product of the magnitudes of the vectors a[n] and ā[n]:

$$θ [ n ] \approx \frac{{\overline{a}}_{x} [ n ] {a}_{z} [ n ] - {\overline{a}}_{z} [ n ] {a}_{x} [ n ]}{‖ a [ n ] ‖ ‖ \overline{a} [ n ] ‖}$$

It should be noted that the tilt angle θ[n] denotes rotation about the y-axis, as the y-axis is depicted in FIG. 1A. In addition, the condition θ_{L}<max(|θ[n]|)<θ_{U} is imposed on the tilt angle θ[n] during the bump interval T_{η}[c]. In an exemplary embodiment, θ_{L} is assigned a value of 0.5° and θ_{U} is assigned a value of 4.0, as these specified values correspond to the exemplary posture recommended for a patient who is being observed using the systems and methods of the present invention. After the angle θ[n] is determined for each point in time in the time interval being analyzed, a signal such as that represented by waveform 73 in FIG. 7 is produced.

Referring now to FIG. 9, a process 500 is used to construct upper and lower frequency bands of the accelerometer signal in order to identify transient features of the measured breathing activity and motions artifacts in the raw accelerometer signal. It should be noted that the process 400 and the process 500 used for preprocessing of the raw accelerometer signal are performed in parallel. At step 501 of process 500, the baseline is removed from the accelerometer signal by applying a third order Butterworth high-pass filter, and in an exemplary embodiment of the disclosed concept, a cutoff frequency of 0.5 Hz is used. At step 502, a third order Butterworth low-pass filter is applied to extract a lower frequency band signal acc_lpf_502, and in an exemplary embodiment, a cutoff frequency of 50 Hz is used. Similarly, at step 503, a third order Butterworth high-pass filter is applied to extract an upper frequency band signal acc_hpf_503, and in an exemplary embodiment, a cutoff frequency of 150 Hz is used. At steps 504 and 505, in order to extract power-like signals from the lower frequency band and upper frequency band, respectively, the lower frequency band signal acc_lpf_502 found at step 502 and the upper frequency band signal acc_hpf_503 found at step 503 are rectified and smoothed using known signal processing techniques. The rectified and smoothed lower frequency band signal produced at step 504 is denoted acc_lpf_sm (produced from acc_lpf_502) and the rectified and smoothed upper frequency band signal produced at step 505 is denoted acc_hpf_sm (produced from acc_hpf_503). As depicted in FIG. 9, step 502 and 504 can be performed in parallel with steps 503 and 505.

It will be appreciated that the steps of process 500 are performed for both the x-channel and the z-channel of accelerometer 4, such that preprocessing the x-channel signal of accelerometer 4 using process 500 produces rectified and smoothed lower and upper frequency band signals acc_lpf_sm_x and acc_hpf_sm_x, while preprocessing the z-channel signal of accelerometer 4 using process 500 produces rectified and smoothed signals acc_lpf_sm_z and acc_hpf_sm_z. Referring again to FIG. 7 in conjunction with FIG. 9, signal 74A is a waveform of an x-channel lower frequency band signal found at step 504 and signal 74B is a waveform of an x-channel upper frequency band signal found at step 505. Similarly, signal 75A is a waveform of a z-channel lower frequency band signal found at step 504 and signal 75B is a waveform of a z-channel upper frequency band signal found at step 505.

Referring now to FIG. 10, a flow chart depicting a process 600 for defining breathing features of the accelerometer signal in terms of the various preprocessed signals found during processes 100-500 is shown. As detailed below, the results of process 500 are used in process 600, and accordingly, it will be appreciated that process 600 is performed on the results of process 500 for both the x-channel and the z-channel. All features found during process 600 are defined over the bump interval T_{η}[c]. At step 601, the smoothed lower frequency band signal acc_lpf_sm produced at step 504 and smoothed upper frequency band signal acc_hpf_sm produced at step 505 are summed. At step 602, the ratio of the smoothed upper frequency band signal produced at step 505 to the summed signal produced at step 601 is found.

Referring again to FIG. 7 in conjunction with FIG. 10, waveform 74A (a lower frequency band signal of the accelerometer 4 x-channel) and waveform 74B (an upper frequency band signal of the accelerometer 4 x-channel) are summed together at step 601 for the x-channel. Similarly, waveform 75A (a lower frequency band signal of the accelerometer 4 z-channel) and waveform 75B (an upper frequency band signal of the accelerometer 4 z-channel) are summed together at step 601 for the z-channel. Waveform 76A results from performing step 602 for the x-channel, as waveform 76A results from finding the ratio of waveform 74B to the sum of waveforms 74A and 74B found at step 601 for the x-channel. Similarly, waveform 76B results from performing step 602 for the z-channel, as waveform 76B results from finding the ratio of waveform 75B to the sum of waveforms 75A and 75B found at step 601 for the z-channel.

In accordance with exemplary embodiments of the present invention, in order for a candidate sniff to qualify as a sniff rather than an artifact, the ratio computed at step 602 associated with the time interval of a candidate sniff must be less than a predetermined value. This requirement is based on the observation that spectograms of accelerometer data typically show that the main power of sniffs occurs in lower frequency regions, whereas the entire frequency band typically contains strong components during artifact activity. Thus, at step 603, if the ratio computed at step 602 is indeed less than the predetermined value, the candidate sniff remains a candidate sniff.

Experimental observations additionally show that, if the upper frequency band signal acc_hpf_sm produced at step 505 crosses a predetermined threshold value more than a predetermined number of times during the time interval of a candidate sniff, the corresponding bump interval T_{η}[c] in the EMG signal is usually due to an artifact. The threshold value is expressed in terms of a fraction of gravitational acceleration (9.8 m/s²). In an exemplary embodiment, the threshold value is chosen to be 0.1 g (i.e. 0.98 m/s²) and the predetermined number of crossings is chosen to be 7. Accordingly at step 604, if the high-frequency band signal acc_hpf_sm crosses the threshold value (e.g. 0.1 g) in excess of the predetermined number of crossings (e.g. 7), the candidate sniff is qualified as artifact, otherwise, the candidate sniff maintains its status as a candidate sniff.

Lastly, at step 605, the variance or standard deviation of both the low-pass filtered and high-pass filtered signals acc_lpf_502 and acc_hpf_503 found during process 500 at steps 502 and 503, respectively, are determined. If any of the variances or standard deviations found at step 605 exceeds a predetermined value, the candidate sniff activity is deemed to correspond to an artifact rather than a sniff. Conversely, if the variances or standard deviations found at step 605 fall below the predetermined value, the candidate sniff is deemed to be a sniff rather than a signal artifact. Waveform 77 in FIG. 7 is an example of a sniff processed EMG signal v[n] for which a number of sniffs 80 have been correctly identified after processes 100-600 have been executed. Referring briefly again to method 50 depicted in FIG. 3, the final step 56 for quantifying RME by finding Eᵣₑₗ is performed after processes 100-600 have been executed for the desired number of breath cycles recorded by EMG electrodes 2 and accelerometer 4, and any sniffs in said breath cycles have been identified.

In an exemplary embodiment of the present invention, it is expected that a true sniff would qualify as a sniff under the criteria of all three of steps 603, 604, and 605, in addition to the criteria previously stated for the EMG-derived features described with respect to processes 100-300 also being satisfied. However, in the event that a candidate sniff exhibited artifact tendencies under the criteria of one or two of steps 603, 604, and 605 but not all three, the predetermined values and threshold values used throughout processes 100-600 may be adjusted without departing from the scope of the disclosed concept. All so-called “threshold values” and “predetermined values” referred to herein are suggested values and are not intended to be limiting on the scope of the present invention. In particular, if methods for preprocessing of EMG signals other than the methods previously stated herein are used, it will be appreciated that at least some of the threshold and predetermined values suggested herein would likely need to be adjusted to account for the differences in magnitude and/or frequency of the preprocessed signal components as compared to those described herein, but that the methods and systems described herein would still be applicable with such adjustments to threshold values and predetermined values.

As previously stated, the methods and processes 50 and 100-600 described herein were developed using a classical engineering and feature extraction approach. Previously, automation of non-invasive sniff detection was challenging and would have resulted in inaccurate distinction between sniffs and signal artifacts in an EMG signal due to the similarities manifested by sniffs and signal artifacts in EMG signals. However, the use of accelerometer signal data in addition to EMG signal data and features by the present invention, particularly processes 400-600, renders automation of accurate and non-invasive distinction between sniffs and signal artifacts possible.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A method of quantifying respiratory effort of a patient during a breathing interval, the method comprising:
measuring, with a number of electromyography (EMG) electrodes, respiratory muscle activity of the patient;
measuring, with an accelerometer, acceleration in a plurality of axes of a thorax of the patient;
receiving, with a controller, a raw EMG signal measured by the EMG electrodes and raw accelerometer signals measured by the accelerometer;
producing a number of preprocessed EMG signals by preprocessing the raw EMG signal with the controller;
producing a number of preprocessed accelerometer signals by preprocessing the raw accelerometer signals with the controller;
identifying, with the controller, portions of the number of preprocessed EMG signals as candidate sniffs;
determining, with the controller, a plurality of EMG-derived features from the number of preprocessed EMG signals associated with time intervals of the candidate sniffs;
determining, with the controller, a plurality of accelerometer signal features from the number of preprocessed accelerometer signals associated with time intervals of the candidate sniffs;
comparing, with the controller, the plurality of EMG-derived features and accelerometer signal features to a plurality of sniff detection threshold values,
classifying, with the controller, the candidate sniffs either as confirmed sniffs or as signal artifacts based on the comparing; and
quantifying, with the controller, a respiratory muscle effort of the patient by comparing a number of attributes of the number of preprocessed EMG signals to a number of attributes of the confirmed sniffs.

2. The method of claim 1, further comprising:
identifying, with the controller, local regular breathing EMG maxima associated with regular breathing in the number of preprocessed EMG signals;
determining the mean of the local regular breathing EMG maxima with the controller; and
identifying, with the controller, a maximum sniff value in the number of preprocessed EMG signals associated with the confirmed sniffs,
wherein quantifying the respiratory muscle effort comprises comparing the mean of the local regular breathing EMG maxima to the maximum sniff value.

3. The method according to claim 1, further comprising:
producing a regular breathing EMG signal by preprocessing the raw EMG signal with the controller to accentuate regular breathing activity and minimize artifacts in the raw EMG signal;
producing a sniff EMG signal by preprocessing the raw EMG signal with the controller to accentuate sniff activity in the raw EMG signal;
identifying, with the controller, the local regular breathing EMG maxima in the regular breathing EMG signal;
identifying, with the controller, local regular breathing EMG minima in the regular breathing EMG signal; and
identifying, with the controller, the maximum sniff value in the sniff EMG signal.

4. The method according to claim 1, wherein quantifying the respiratory muscle effort of the patient comprises finding a ratio of the mean of the local EMG maxima to the maximum sniff value.

5. The method of claim 3, further comprising:
identifying for each candidate sniff, with the controller, a bump of the candidate sniff such that all values of the sniff EMG signal in the bump are greater than or equal to a predetermined threshold sniff value;
identifying, with the controller, a midpoint in each bump, said midpoint being a median such that an area under a curve of a left half of the bump is equal to an area under a curve of a right half of the bump;
calculating at the midpoint of each bump, with the controller, an offset value by linearly interpolating between the local sniff EMG minimum immediately preceding the bump and the local sniff EMG minimum immediately following the bump;
determining, with the controller, an amplitude of the bump by finding the difference between a maximum sniff EMG value in the bump and the offset value; and
classifying the candidate sniff as an artifact if a number of predetermined amplitude conditions are indicative of artifact activity.

6. The method according to claim 5, further comprising:
determining a first asymmetry feature of the bump, with the controller, by finding the ratio of the mean of the local sniff EMG minimum immediately preceding the bump and the local sniff EMG minimum immediately following the bump to the amplitude;
determining a second asymmetry feature of the bump, with the controller, by finding a first difference between the local sniff EMG minimum immediately preceding the bump and the local sniff EMG minimum immediately following the bump, by finding a second difference between the maximum sniff value in the bump and the local sniff EMG minimum immediately preceding the bump, by finding a third difference between the maximum sniff value in the bump and the local sniff EMG minimum immediately following the bump, and by finding the ratio of the first difference to the lesser of the second difference and the third difference;
determining a third asymmetry feature of the bump, with the controller, by determining a skewness of the bump; and
classifying the candidate sniff as an artifact if a number of predetermined asymmetry conditions are indicative of artifact activity.

7. The method according to claim 1, further comprising:
low-pass filtering, rectifying, and smoothing the raw accelerometer signals with the controller to produce a lower frequency band power signal;
high-pass filtering, rectifying, and smoothing the raw accelerometer signals with the controller to produce an upper frequency band power signal,
summing the lower frequency band power signal and upper frequency band power signal with the controller to produce a summed frequency band power signal;
determining a first ratio of the upper frequency band power signal to the summed frequency band power signal for a first axis of the accelerometer during the time interval associated with each of the candidate sniffs;
determining a second ratio of the upper frequency band power signal to the summed frequency band power signal for a second axis of the accelerometer during the time interval associated with each of the candidate sniffs;
comparing the first ratio and the second ratio to a predetermined frequency band ratio with the controller; and
qualifying as an artifact any of the candidate sniffs for which the first ratio and the second ratio exceeds the predetermined frequency band ratio.

8. The method (s) according to claim 1, further comprising:
high-pass filtering, rectifying, and smoothing the raw accelerometer signals with the controller to produce an upper frequency band power signal;
determining, with the controller, the number of times the upper frequency band power signal crosses a predetermined threshold value during the time interval associated with each of the candidate sniffs; and
qualifying as an artifact, with the controller, any of the candidate sniffs for which the number of times the upper frequency band power signal crosses the predetermined threshold exceeds a predetermined number of crossings during the time interval associated with the candidate sniff.

9. The method according to claim 1, further comprising:
low-pass filtering the raw accelerometer signals with the controller to produce a lower frequency band signal;
high-pass filtering the raw accelerometer signals with the controller to produce an upper frequency band signal;
determining, with the controller, the standard deviation of the lower frequency band signal and the higher frequency band signal; and
qualifying as an artifact, with the controller, any of the candidate sniffs for which the standard deviation exceeds a predetermined value.

10. A system for quantifying respiratory effort of a patient during a breathing interval, the system comprising:
a number of electromyography (EMG) electrodes configured to measure respiratory muscle activity of the patient;
an accelerometer configured to measure acceleration in a plurality of axes of a thorax of the patient; and
a controller,
wherein the controller is configured to receive a raw EMG signal measured by the EMG electrodes and raw accelerometer signals measured by the accelerometer,
wherein the controller is configured to produce a number of preprocessed EMG signals by preprocessing the raw EMG signal,
wherein the controller is configured to produce a number of preprocessed accelerometer signals by preprocessing the raw accelerometer signals,
wherein the controller is configured to identify portions of the number of preprocessed EMG signals as candidate sniffs,
wherein the controller is configured to determine a plurality of EMG-derived features from the number of preprocessed EMG signals associated with time intervals of the candidate sniffs,
wherein the controller is configured to determine a plurality of accelerometer signal features from the number of preprocessed accelerometer signals associated with time intervals of the candidate sniffs,
wherein the controller is configured to compare the plurality of EMG-derived features and accelerometer signal features to a plurality of sniff detection threshold values,
wherein the controller is configured to classify the candidate sniffs as confirmed sniffs or as signal artifacts based on comparisons of the plurality of EMG-derived features and accelerometer signal features to the plurality of sniff detection threshold values, and
wherein the controller is configured to quantify a respiratory muscle effort of the patient by comparing a number of attributes of the number of preprocessed EMG signals to a number of attributes of the confirmed sniffs.

11. The system of claim 10, wherein the controller is further configured to:
identify local regular breathing EMG maxima associated with regular breathing in the number of preprocessed EMG signals;
determine the mean of the local regular breathing EMG maxima;
identify a maximum sniff value in the number of preprocessed EMG signals associated with the confirmed sniffs; and
quantify the respiratory muscle effort by comparing the mean of the local EMG maxima to the maximum sniff value.

12. The system according to claim 10, wherein the controller is further configured to:
produce a regular breathing EMG signal by preprocessing the raw EMG signal to accentuate regular breathing activity and minimize artifacts in the EMG signal;
produce a sniff EMG signal by preprocessing the raw EMG signal to accentuate sniff activity in the EMG signal;
identify local EMG maxima in the regular breathing EMG signal; and
identify the maximum sniff value in the sniff EMG signal, and
wherein comparing the mean of the local EMG maxima to the maximum sniff value comprises finding the ratio of the mean of the local EMG maxima to the maximum sniff value.

13. The system of claim 12, wherein the controller is further configured to:
identify, for each candidate sniff, a bump of the candidate sniff such that all values of the sniff EMG signal in the bump are greater than or equal to a predetermined threshold sniff value;
identify a midpoint in each bump, said midpoint being a median such that an area under a curve of a left half of the bump is equal to an area under a curve of a right half of the bump;
determine, at the midpoint of each bump, an offset value by linearly interpolating between the local sniff EMG minimum immediately preceding the bump and the local sniff EMG minimum immediately following the bump;
determine an amplitude of the bump by finding the difference between a maximum sniff EMG value in the bump and the offset value; and
classify the candidate sniff as an artifact if a number of predetermined amplitude conditions are indicative of artifact activity.

14. The system according to claim 10, wherein the controller is further configured to:
high-pass filter, rectify, and smooth the raw accelerometer signals to produce an upper frequency band power signal;
determine the number of times the upper frequency band power signal crosses a predetermined threshold value during the time interval associated with each of the candidate sniffs; and
qualify as an artifact any of the candidate sniffs for which the number of times the upper frequency band power signal crosses the predetermined threshold exceeds a predetermined number of crossings during the time interval associated with the candidate sniff.

15. The system according to claim 10, wherein controller is further configured to:
low-pass filter the raw accelerometer signals to produce a lower frequency band signal;
high-pass filter the raw accelerometer signals to produce an upper frequency band signal;
determine the standard deviation of the lower frequency band signal and the higher frequency band signal; and
qualify as an artifact any of the candidate sniffs for which the standard deviation exceeds a predetermined value.
